(19) United States
(12) Patent Application Publication
(10) Pub. No.: US 2022/0125328 A1
(43) Pub. Date: Apr. 28, 2022

(54) TIME FEATURES CALCULATION APPARATUS, CALCULATION METHOD AND ITS PROGRAM

(71) Applicant: NIPPON TELEGRAPH AND TELEPHONE CORPORATION, Tokyo (JP)

(72) Inventors: Kana EGUCHI, Musashino-shi, Tokyo (JP); Ryosuke AOKI, Musashino-shi, Tokyo (JP); Shigekuni KONDO, Musashino-shi, Tokyo (JP); Takuya INDO, Musashino-shi, Tokyo (JP)

(73) Assignee: NIPPON TELEGRAPH AND TELEPHONE CORPORATION, Tokyo (JP)

(21) Appl. No.: 17/430,360

(22) PCT Filed: Jan. 31, 2020
(86) PCT No.: PCT/JP2020/003579

§ 371 Date: Aug. 12, 2021

(30) Foreign Application Priority Data
Feb. 14, 2019 (JP) 2019-024645

(51) Int. Cl. A61B 5/024; A61B 5/0205; A61B 5/08

(52) U.S. Cl. CPC A61B 5/02405; A61B 5/0816; A61B 5/0205

(57) ABSTRACT

A time feature value calculation apparatus according to an embodiment includes an interpolation unit (182) and a time feature value calculation unit (183). In accordance with a variability tendency of a biological signal exhibited in time-series data of the biological signal resulting from an abnormality value process performed to eliminate an abnormality value from the biological signal in response to an input of the biological signal having periodicity, the interpolation unit interpolates a missing value in the biological signal in the time-series data of the biological signal resulting from the abnormality value process. The time feature value calculation unit calculates a time feature value focusing on the variability tendency of the biological signal measured over an arbitrary time period, from the interpolated time-series data of the biological signal.

### TECHNICAL FIELD

The present invention relates to a time feature value calculation apparatus, a calculation method, and a program therefor.

### BACKGROUND ART

[Outline of Heart Rate Variability Analyses in General]

The autonomic nerves include the sympathetic nerves and the vagus nerve. Both types of nerves are widely distributed in organs and the like and control involuntary physical functions such as circulation and metabolism. It is said that, in many situations, both types of nerves control each organ antagonistically.

It is known that sympathetic nerve activities, which are one type of autonomic nerve activities, are enhanced by stress stimulation such as mental calculation stress.

The vagus nerve, which is the other type of autonomic nerve, is primarily in charge of parasympathetic nerve activities in each of the organs controlled by the vagus nerve. Accordingly, the vagus nerve activities are often recognized as being equivalent to parasympathetic nerve activities. The term “vagus nerve” is, strictly speaking, the name of the X-th nerve, which is one of the cranial nerves, and denotes the entirety of this nerve extending from the brain to various organs. For this reason, the parasympathetic nerve activities in an organ in question may be referred to by appending the name of the organ being controlled (e.g., the cardiac vagus nerve).

Examples of the organs controlled by the autonomic nerves include the heart. The heart is antagonistically controlled by a sympathetic nerve and the vagus nerve and is said to reflect static balance between the activities of the two types of autonomic nerves (see reference document [i]).

In particular, it is known that fluctuation of instantaneous heart rates (R-R intervals [RRIs]) each expressed as an interval between two adjacent R-waves is varied by the activities of the two types of autonomic nerves. R-waves are a type of electrocardiographic waveform obtained by acquiring an electrocardiogram and reflect depolarization activities of the heart (see reference document [ii]).

As a method for estimating autonomic nerve activities in real environment, heart rate variability analyses are known. Heart rate variability analyses can roughly be categorized into time feature value analyses and frequency feature value analyses (see reference document [iii]).

[Calculating Time Feature Values of Heart Rates]

In the time feature value analyses, a temporal feature value is calculated with respect to instantaneous heart rates represented by irregular intervals, on the basis of a condition or an expression determined for each feature value (see reference document [iii]). Time feature values of instantaneous heart rates can roughly be categorized into two types: one focusing on a relationship between two adjacent instantaneous heart rates; and the other focusing on a variability tendency of instantaneous heart rates measured over an arbitrary time period.

Examples of time feature values focusing on a relationship between adjacent instantaneous heart rates include: the square root of the mean square value of temporal differences between adjacent RRIs (RMSSD); the standard deviation of temporal differences between adjacent normal RRIs (SDSD); a total number of pairs of adjacent RRIs between which the temporal difference exceeds 50 milliseconds (NN50); the percentage of the number of times of occurrence where the temporal difference between a pair of adjacent RRIs is equal to or longer than 50 milliseconds (pNN50); a parasympathetic nerve activity index calculated from the length L of the long side and the length T of the short side of a shape drawn by a Lorenz plot of adjacent RRIs (CVI); and a sympathetic nerve activity index calculated from L and T (CSI).

Examples of time feature values focusing on a variability tendency of instantaneous heart rates measured over an arbitrary time period include: the standard deviation of RRIs (SDNN); the standard deviation of an average RRI over a prescribed time period (SDANN); a variability coefficient of RRIs (CVNN); an average RRI, and an average heart rate.

Because of these calculation processes, it is possible to calculate the time feature values by using time-series data of a relatively small number of instantaneous heart rates, compared to the frequency feature values. It is often the case that a calculation interval is set in accordance with the purpose of use.

[Calculating Frequency Feature Values of Heart Rates]

In the frequency feature value analyses, a low frequency component from a frequency spectrum analysis is interpreted as an index reflecting sympathetic nerve activities and cardiac vagus nerve activities, whereas a high frequency component is interpreted as an index reflecting cardiac vagus nerve activities (see reference documents [i] and [iii]).

Among feature values of heart rates, to calculate a frequency feature value, it is necessary to calculate power spectral density after performing a re-sampling process so that instantaneous heart rates, which are irregular intervals, are represented by data at regular intervals (see reference document [i]).

Because frequency bands to be focused on as a low frequency component and a high frequency component are determined in advance and because of relationships among various types of physiological indices that can impact frequency feature values of heart rates, frequency feature values require time-series data of instantaneous heart rates over a relatively long period of time, compared to time feature values. Generally speaking, to evaluate autonomic nerve functions in a physiologically stable state, data over a five-minute period is used (see reference document [i]). Even when a transient autonomic nerve reaction is to be grasped, it is considered necessary to have data corresponding to approximately 100 heartbeats in order to extract a low frequency component and a high frequency component (see reference document [i]).

[Outline of Electrocardiogram Acquisition and Measured Signals]

Examples of means for acquiring electrocardiograms include wearable devices such as Holter electrocardiographs. The electrocardiograms obtained by using these devices may have measurement abnormalities due to various causes such as electrode abnormalities including deformations and shifts of electrodes, as well as body movements, perspiration, and static electricity (see reference document [iv]). The measurement abnormalities can be recognized in electrocardiograms as artifacts or noise. The sustained durations of both the noise and the artifacts vary depending on lasting time periods of the measurement abnormalities.

A waveform observed as an artifact has a frequency characteristic that closely resembles to that of an R-wave. It is therefore extremely difficult to completely remove artifacts by using a commonly-used filtering method. For this reason, in some situations, algorithms designed to analyze electrocardiograms to extract R-waves may erroneously determine and extract an artifact as an R-wave.

The time feature values and the frequency feature values are based on the premise that all of the data subject to an analysis represents normal instantaneous heart rates. In this situation, the “normal” state denotes a state in which neither the measured subject nor the measurement device has abnormalities. Abnormalities of the measured subject include arrhythmia. Abnormalities of the measurement device denote situations in which a measurement abnormality has occurred in the electrocardiogram.

As one of the measurement abnormalities, an artifact being erroneously determined as an R-wave does not reflect the depolarization activities of the heart at all due to the developmental mechanism thereof. For this reason, when the R-waves structuring the instantaneous heart rates subject to an analysis include at least one artifact erroneously determined as an R-wave, neither time feature values nor frequency feature values are considered to reflect autonomic nerve activities.

Accordingly, to prevent artifacts from being erroneously determined as R-waves, the following methods have been proposed.

[Method 1]

As a method for analyzing variability of heart rates by using an electrocardiogram acquired by a wearable device, Non-Patent Literature 1 and Non-Patent Literature 2 each propose a method by which, prior to the calculation of a heart rate feature value, abnormality values are eliminated from instantaneous heart rates on the basis of time features of the instantaneous heart rates. Further, Non-Patent Literature 3 proposes a method by which, prior to the calculation of a heart rate feature value, abnormality values are eliminated from instantaneous heart rates on the basis of the measurement state of the instantaneous heart rates and time features. Using any of these abnormality value elimination methods is expected to improve the precision level of the calculation of the time feature value by appropriately eliminating the abnormality values from the instantaneous heart rates.

However, when a frequency feature value is calculated, if only Method 1 above is used, it is also known that the frequency component greatly varies, and the feature value cannot be calculated correctly (see Non-Patent Literatures 4 and 5).

[Method 2]

As a method for calculating a frequency feature value by using an electrocardiogram acquired by a wearable device, which may perhaps be found insufficient when only Method 1 described above is used, Non-Patent Literature 4 proposes a method by which missing values are supplemented with a direct current component for the purpose of suppressing impacts of the missing values. In addition, Non-Patent Literature 5 proposes a complement method using a non-linear function estimated from time-series data of instantaneous heart rates measured normally.

### CITATION LIST

### Non-Patent Literature

Non-Patent Literature 1: Sakuma, D. et al., A Real-Time Relaxation System at Sitting Position Using Heart Rate Measurement, Papers from Multimedia Bunsan Kyocho to Mobile Symposium 2013 [The 2013 Symposium on Multimedia, Decentralization, Collaboration, and Mobile (in Japanese)], pp. 1188-1195, 2013

Non-Patent Literature 2: Yokota, Y. et al., Monitoring of Sepsis Premonitories Using Time Series of Heart Rate Variability, The 54th Japan Joint Automatic Control Conference, pp. 1258-1261, 2011

Non-Patent Literature 3: Eguchi, K. et al., Reliability Evaluation of R-R Interval Measurement Status Using the Electric Potential Characteristics of QRS Complex for Wearable ECG Devices, IEICE Technical Report, vol. 116, no. 412, pp. 171-176, 2017

Non-Patent Literature 4: Eguchi, K. et al., Missing R-R Interval Complement Method for Heart Rate Variability Analysis in Frequency Dominant Using Wearable ECG Devices, Papers from Multimedia Bunsan Kyocho to Mobile Symposium 2017 [The 2017 Symposium on Multimedia, Decentralization, Collaboration, and Mobile (in Japanese)], pp. 888-897, 2017

Non-Patent Literature 5: Eguchi, K. et al., R-R Interval Outlier Processing for Heart Rate Variability Analysis Using Wearable ECG Devices, Advanced Biomedical Engineering, vol. 7, pp. 28-38, 2018

### SUMMARY OF THE INVENTION

### Technical Problem

When abnormality values are eliminated by using Method 1 described above, as a result, the instantaneous heart rates in a section free from measurement abnormalities may, in some situations, remain as normal values. When a time feature value focusing on a variability tendency of the instantaneous heart rates is calculated from such instantaneous heart rates, the instantaneous heart rates remaining as a result of the abnormality value elimination process according to Method 1 is not able, by itself, to reflect the variability tendency, and the precision level of the calculation may be degraded, contrary to the expectation, in some situations.

Further, among various time feature values of heart rates, time feature values focusing on the relationship between adjacent instantaneous heart rates are suitable for the calculation eliminating abnormality values, in view of the original definition of the feature values as well. It is therefore considered that Method 1 described above will have no problem.

Further, Method 2 described above aims to suppress the errors in the calculation of the power spectral density performed after the data re-sampling at the time of calculating a frequency feature value of the heart rates. For this reason, even when Method 2 is applied to the calculation of a time feature value focusing on an instantaneous heart rate variability tendency, it may be difficult to appropriately reflect the instantaneous heart rate variability tendency. Thus, there is a possibility that the precision level of the calculation of the feature value may not be improved.

In particular, of Method 2 described above, a complement method using a non-linear function such as that proposed in Non-Patent Literature 5 is based on the premise where, generally speaking, a certain data length is required in the calculation of a frequency feature value. For this reason, even when this method is applied to the calculation of a time feature value focusing on an instantaneous heart rate variability tendency, there is a possibility that it may be impossible to ensure a sufficient length of time-series data required by this method of the instantaneous heart rates, depending on the calculation interval of the time feature value. In addition, when the calculation interval of the time feature value is adjusted in favor of application of this method, there is also a possibility that a delay may be caused by the time required by the calculation of the time feature value of the heart rates, which may make it impossible to grasp the instantaneous heart rate variability, which was the original purpose. In addition, it is known that heart rate variability tendencies and frequency characteristics do not necessarily match (see reference document [i]). Accordingly, the time-series data of instantaneous heart rates obtained from a complement method using a non-linear function such as that described in Non-Patent Literature 5 may not necessarily be capable of grasping the variability tendency of the instantaneous heart rates.

In view of the circumstances described above, it is an object of the present invention to provide a time feature value calculation apparatus, a calculation method, and a program therefor capable of calculating a time feature value with excellent precision focusing on a variability tendency of a biological signal measured over an arbitrary time period, from time-series data of the biological signal which has periodicity like that of instantaneous heart rates and which has a missing section caused by a measurement abnormality or the like.

### Means for Solving the Problem

A first mode of the present invention provides a time feature value calculation apparatus including: an interpolation unit that, in accordance with a variability tendency of a biological signal having periodicity exhibited in time-series data of the biological signal having periodicity resulting from an abnormality value process performed to eliminate an abnormality value from the biological signal having periodicity in response to an input of the biological signal having periodicity, interpolates a missing value in the biological signal having periodicity in the time-series data of the biological signal having periodicity resulting from the abnormality value process; and a time feature value calculation unit that calculates a time feature value focusing on the variability tendency of the biological signal having periodicity measured over an arbitrary time period, from the time-series data of the biological signal having periodicity interpolated by the interpolation unit.

A second mode of the present invention is based on the first mode in which the interpolation unit interpolates the missing value in the biological signal having periodicity, by performing one selected from among: a linear interpolation; an interpolation using a mathematical function approximating the variability tendency of the biological signal having periodicity; and an interpolation using an average value of data sectioned by an arbitrary duration.

A third mode of the present invention is based on the first mode further including: a calculation target determination unit that determines whether a characteristic of the time feature value to be calculated corresponds to a time feature value focusing on the variability tendency of the biological signal having periodicity measured over the arbitrary time period or to a time feature value focusing on a relationship between adjacent signals each being the biological signal having periodicity. The interpolation unit operates only when the calculation target determination unit determines that the characteristic of the time feature value to be calculated includes the time feature value focusing on the variability tendency of the biological signal having periodicity measured over the arbitrary time period.

A fourth mode of the present invention is based on the first mode further including: a calculation target determination unit that determines whether or not the interpolation by the interpolation unit is to be performed, depending on a type of the time feature value which is to be calculated by the time feature value calculation unit and which focuses on the variability tendency of the biological signal having periodicity measured over the arbitrary time period. When the calculation target determination unit determines that the interpolation by the interpolation unit is not to be performed, the time feature value calculation unit calculates the time feature value focusing on the variability tendency of the biological signal having periodicity measured over the arbitrary time period, from the time-series data of the biological signal having periodicity resulting from the process to eliminate the abnormality value.

A fifth mode of the present invention is based on the fourth mode in which the biological signal having periodicity is an instantaneous heart rate, while examples of the type of the time feature value which is to be calculated by the time feature value calculation unit and which focuses on the variability tendency of the biological signal having periodicity measured over the arbitrary time period include an average heart rate, an average RRI, CVNN, SDNN, and SDANN. The calculation target determination unit determines that the interpolation by the interpolation unit is to be performed, when the time feature value to be calculated is one of the average RRI and the average heart rate.

A sixth mode of the present invention provides a time feature value calculation method for calculating a time feature value of a biological signal having periodicity from time-series data of the biological signal having periodicity, including: interpolating, in accordance with a variability tendency of the biological signal having periodicity exhibited in the time-series data of the biological signal having periodicity resulting from an abnormality value process performed to eliminate an abnormality value from the biological signal having periodicity in response to an input of the biological signal having periodicity, a missing value in the biological signal having periodicity in the time-series data of the biological signal having periodicity resulting from the abnormality value process; and calculating a time feature value focusing on the variability tendency of the biological signal having periodicity measured over an arbitrary time period, from the interpolated time-series data of the biological signal having periodicity.

A seventh mode of the present invention provides a program that, when executed by a computer, causes the computer to function as the time feature value calculation apparatus according to any one of the first to the fifth modes.

### Effects of the Invention

According to the present invention, it is possible to provide the time feature value calculation apparatus, the calculation method, and the program therefor capable of calculating, with excellent precision, the time feature value focusing on the variability tendency of the biological signal having periodicity measured over the arbitrary time period, from the time-series data of the biological signal having periodicity that has the missing section caused by a measurement abnormality or the like.

### BRIEF DESCRIPTION OF DRAWINGS

FIG. 1 is a diagram showing a configuration of a heart rate variability analysis system employing an instantaneous heart rate time feature value calculation apparatus according to an embodiment of a time feature value calculation apparatus of the present invention.

FIG. 2 is a chart showing a relationship between R-waves and an instantaneous heart rate (RRI) in an electrocardiogram.

FIG. 3 is a chart showing examples of measurement abnormalities (artifacts and noise) in an electrocardiogram.

FIG. 4 is a functional block diagram of the instantaneous heart rate time feature value calculation apparatus according to the embodiment of the present invention.

FIG. 5 is a drawing showing a flowchart for explaining an operation of the instantaneous heart rate time feature value calculation apparatus.

FIG. 6A is a chart showing an example of time-series data of instantaneous heart rates before measurement abnormality values are eliminated.

FIG. 6B is a chart showing an example of the time-series data of the instantaneous heart rates after the measurement abnormality values are eliminated.

FIG. 7 is a flowchart for explaining a process at step S16 in FIG. 5.

FIG. 8A is a chart showing an example of a linear interpolation which is performed by an instantaneous heart rate interpolation unit in FIG. 4 and is based on values before and after a missing value.

FIG. 8B is a chart showing an example of an interpolation which is performed by the instantaneous heart rate interpolation unit in FIG. 4 and which uses a linear function or the like approximating an RRI variability tendency measured over a prescribed time period.

FIG. 8C is a chart showing an example of an interpolation which is performed by the instantaneous heart rate interpolation unit in FIG. 4 and which uses an average value of pieces of data sectioned by an arbitrary duration.

FIG. 8D is a chart showing another example of the interpolation which is performed by the instantaneous heart rate interpolation unit in FIG. 4 and which uses an average value of pieces of data sectioned by an arbitrary duration.

FIG. 9 is a flowchart for explaining a modified part of the process at step S16 according to a modification example.

FIG. 10 is a chart showing a P-wave interval (PPI).

FIG. 11 is a chart showing a respiration cycle.

### DESCRIPTION OF EMBODIMENTS

The following will describe a heart rate variability analysis system employing an instantaneous heart rate time feature value calculation apparatus according to an embodiment of a time feature value calculation apparatus of the present invention, with reference to the drawings.

[Configuration]

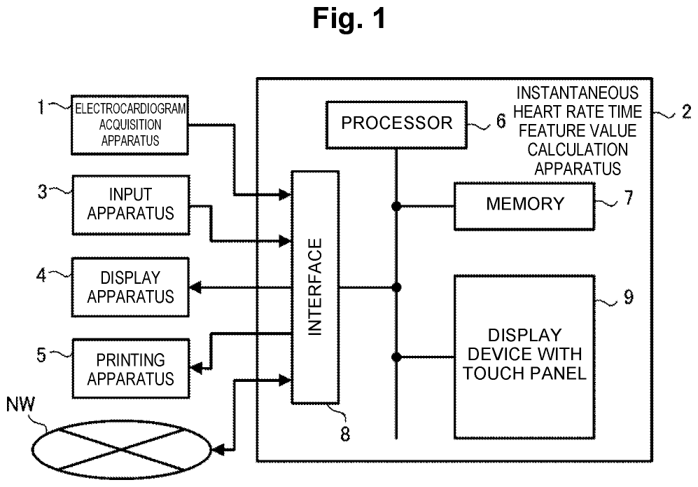

FIG. 1 is a diagram showing a configuration of a heart rate variability analysis system. The heart rate variability analysis system includes an electrocardiogram acquisition apparatus 1 and an instantaneous heart rate time feature value calculation apparatus 2 according to an embodiment of the present invention. Further, the heart rate variability analysis system may include at least one selected from among: an input apparatus 3, a display apparatus 4, and a printing apparatus 5.

The electrocardiogram acquisition apparatus 1 acquires an electrocardiogram of a subject and sends the electrocardiogram to the instantaneous heart rate time feature value calculation apparatus 2. The electrocardiogram acquisition apparatus 1 acquires the electrocardiogram by using electrodes corresponding to at least two poles. The electrocardiogram indicates chronological changes in a biological signal of a circulatory system, e.g., in a periodic signal synchronized with contraction and expansion of the heart chambers. In other words, the electrocardiogram includes time-series data from which it is possible to extract electrocardiographic information corresponding to R-waves reflecting a depolarization activity of the heart.

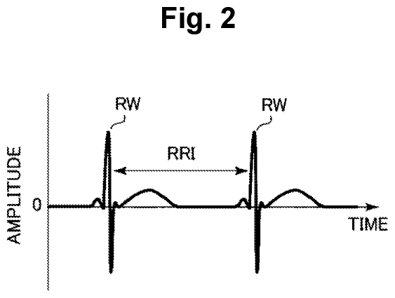

FIG. 2 is a chart showing a relationship between R-waves and an instantaneous heart rate (RRI) in an electrocardiogram. As shown in FIG. 2, the electrocardiogram is expressed as chronological changes in electric potential measured by the abovementioned electrodes corresponding to at least two poles and includes R-waves RW reflecting the depolarization activity of the heart. The interval between two adjacent R-waves is an instantaneous heart rate (RRI).

As long as it is possible to acquire electrocardiographic information corresponding to R-waves, the mode for realizing the electrocardiogram acquisition apparatus 1 is not limited. For example, the electrocardiogram acquisition apparatus 1 may be formed as a wearable device such as a Holter electrocardiograph that can be worn by the subject. Further, the electrocardiogram acquisition apparatus 1 may integrally be formed with the instantaneous heart rate time feature value calculation apparatus 2. In other words, the heart rate variability analysis system may be realized as one wearable device. In another example, the electrocardiogram acquisition apparatus 1 may be provided outside the heart rate variability analysis system. In other words, the heart rate variability analysis system may be configured so that a result of acquiring the electrocardiogram of the subject is input from an external apparatus corresponding to the electrocardiogram acquisition apparatus 1, to the instantaneous heart rate time feature value calculation apparatus 2, via a network NW such as the Internet.

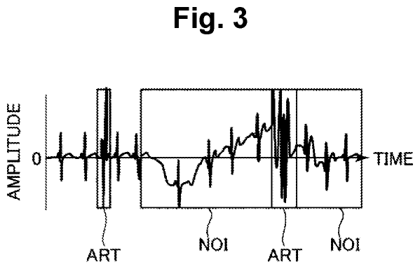

Further, when the electrocardiogram acquisition apparatus 1 is formed as a wearable device, the electrocardiogram may have measurement abnormalities due to various causes such as electrode abnormalities including deformations and shifts of electrodes, as well as body movements, perspiration, and static electricity. FIG. 3 is a chart showing examples of measurement abnormalities in an electrocardiogram. In other words, the measurement abnormalities can be recognized in the electrocardiogram as artifacts ART and noise NOI indicated in the drawing. The sustained durations of both the noise NOI and the artifacts ART vary depending on lasting time periods of the measurement abnormalities.

The instantaneous heart rate time feature value calculation apparatus 2 receives an input of the electrocardiogram acquired by the electrocardiogram acquisition apparatus 1 and calculates time feature values of instantaneous heart rates. The instantaneous heart rate time feature value calculation apparatus 2 may be realized by using, for example, a computer device such as a smartphone, a tablet terminal, or a personal computer (PC). When being realized with a smartphone, the instantaneous heart rate time feature value calculation apparatus 2 includes a processor 6 such as a Central Processing Unit (CPU), a memory 7 connected to the processor 6, an interface 8 used for communicating with the electrocardiogram acquisition apparatus 1 in a wireless or wired manner, and a display device with touch panel 9. As a result of executing a program stored in the memory 7, the processor 6 is able to obtain the electrocardiogram from the electrocardiogram acquisition apparatus 1 via the interface 8, to receive a designation of a time feature value to be calculated (hereinafter, “calculation target”) through a user operation performed on the display device with touch panel 9, to calculate the time feature value of the instantaneous heart rates corresponding to the designation from the obtained electrocardiogram, and to have the calculation result displayed on the display device with touch panel 9. In this situation, the user may be the subject himself/herself or may be someone other than the subject such as a medical doctor or a researcher who makes assessment based on the time feature value of the instantaneous heart rates calculated by the instantaneous heart rate time feature value calculation apparatus 2. In another example, when the instantaneous heart rate time feature value calculation apparatus 2 is realized by using a PC, it is possible to use the input apparatus 3 including a keyboard and a pointing device such as a mouse and the display apparatus 4 such as a liquid crystal display monitor, in place of the display device with touch panel 9. In that situation, the interface 8 is provided with a function to communicate with the input apparatus 3 and the display apparatus 4 in a wireless or wired manner. The interface 8 may further be provided with a function to communicate with the printing apparatus 5 such as a printer in a wireless or wired manner. Further, the interface 8 may be provided with a function to communicate with other devices in the network NW such as the Internet or a local Area Network (LAN), by connecting to the network NW in a wireless or wired manner.

In this situation, the mode for realizing the instantaneous heart rate time feature value calculation apparatus 2 is not limited to the one in the present example. The instantaneous heart rate time feature value calculation apparatus 2 does not necessarily have to include the display device with touch panel 9. Also, the instantaneous heart rate time feature value calculation apparatus 2 does not necessarily have to be connected to one or more or to any of: the input apparatus 3, the display apparatus 4, the printing apparatus 5, and the network NW. For example, the designation of the time feature value to be calculated (the calculation target) may be a designation indicated in a certain setting file or a variable stored in the memory 7. Further, for example, when the time feature value is calculated as an input value to a certain machine learning algorithm, the instantaneous heart rate time feature value calculation apparatus 2 may forward only the calculated time feature value to a machine learning apparatus (not shown) as the input value to the machine learning algorithm, without visualizing the calculated time feature value. In other words, the instantaneous heart rate time feature value calculation apparatus 2 may be connected to such an apparatus that uses the calculated time feature value as an input apparatus for the apparatus or may be incorporated as a part of the apparatus.

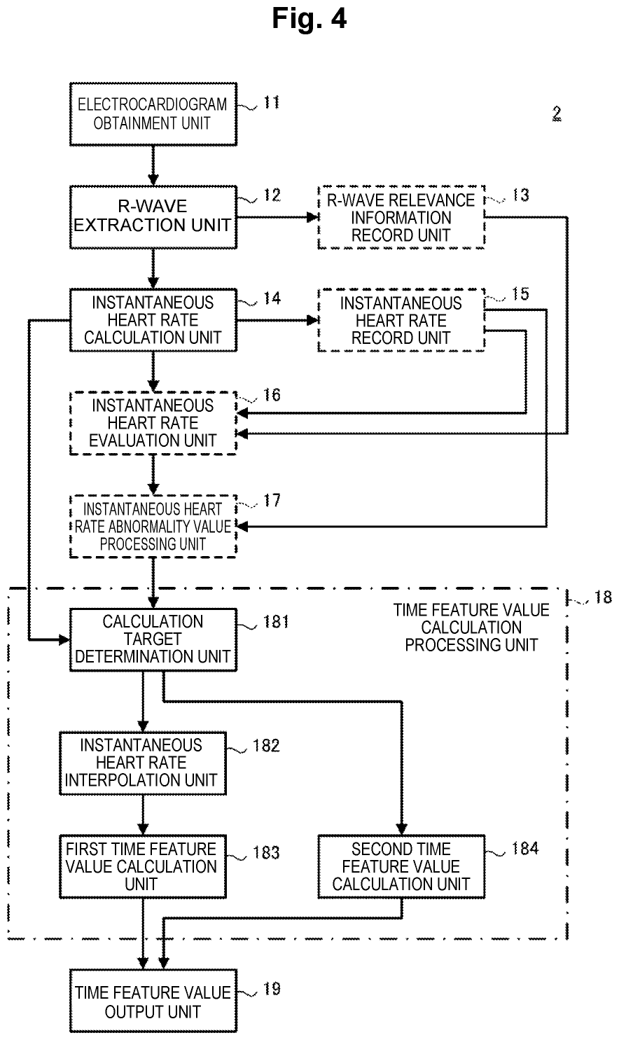

FIG. 4 is a functional block diagram of the instantaneous heart rate time feature value calculation apparatus 2. The instantaneous heart rate time feature value calculation apparatus 2 includes an electrocardiogram obtainment unit 11, an R-wave extraction unit 12, an R-wave relevance information record unit 13, an instantaneous heart rate calculation unit 14, an instantaneous heart rate record unit 15, an instantaneous heart rate evaluation unit 16, an instantaneous heart rate abnormality value processing unit 17, a time feature value calculation processing unit 18, and a time feature value output unit 19. The functions of the electrocardiogram obtainment unit 11, the R-wave extraction unit 12, the R-wave relevance information record unit 13, the instantaneous heart rate calculation unit 14, the instantaneous heart rate record unit 15, the instantaneous heart rate evaluation unit 16, the instantaneous heart rate abnormality value processing unit 17, the time feature value calculation processing unit 18, and the time feature value output unit 19 are realized as a result of the processor 6 reading and executing the program stored in the memory 7. A part or all of these functions may be realized by a circuit such as an Application Specific Integrated Circuit (ASIC) or a Field Programmable Gate Array (FPGA).

The electrocardiogram obtainment unit 11 obtains the electrocardiogram acquired by the electrocardiogram acquisition apparatus 1. More specifically, the processor 6 obtains the electrocardiogram from the electrocardiogram acquisition apparatus 1 via the interface 8 and stores the electrocardiogram into the memory 7. Alternatively, the processor 6 may receive the electrocardiogram acquired by an external apparatus corresponding to the electrocardiogram acquisition apparatus 1 from the apparatus or from a server saving therein the acquired electrocardiogram, via the network NW through the interface 8, so as to store the received electrocardiogram into the memory 7. Further, although not shown in the drawings particularly, when the interface 8 has a function to read from a removable medium (a memory card) detachably attached to the instantaneous heart rate time feature value calculation apparatus 2, the processor 6 may obtain the electrocardiogram acquired by either the electrocardiogram acquisition apparatus 1 or an external apparatus corresponding to the electrocardiogram acquisition apparatus 1 via the storage medium.

The R-wave extraction unit 12 analyzes the electrocardiogram stored in the memory 7 and extracts the R-waves. In the embodiment, the specific method for extracting the R-waves is not limited. If required in a subsequent process, information relevant to the extracted R-wave is recorded in the R-wave relevance information record unit 13.

In the situation where, for example, the instantaneous heart rate evaluation unit 16 performs a process of eliminating abnormality values other than the instantaneous heart rates, by using a publicly-known technique, the R-wave relevance information record unit 13 records information related to the R-waves extracted by the R-wave extraction unit 12, such as electric potentials of the R-waves, for example. Although this function is not a requisite function of the embodiment, this function is necessary when the instantaneous heart rate evaluation unit 16 evaluates a measurement state on the basis of a measurement state of the R-waves. The present embodiment example is based on the situation where the measurement state is evaluated on the basis of the measurement state of the R-waves. The R-wave relevance information record unit 13 may be configured by using either the memory 7 or another recording medium that is not shown. In the embodiment, the specific information recording method used by the R-wave relevance information record unit 13 is not particularly specified.

The instantaneous heart rate calculation unit 14 calculates an instantaneous heart rate on the basis of the R-waves extracted by the R-wave extraction unit 12 and saves calculated instantaneous heart rate information into the instantaneous heart rate record unit 15.

The instantaneous heart rate record unit 15 records the instantaneous heart rates calculated by the instantaneous heart rate calculation unit 14. The instantaneous heart rate record unit 15 may be configured by using the memory 7 or another recording medium (not shown). In the embodiment, the specific information record format used by the instantaneous heart rate record unit 15 is not particularly specified. In an example, the information may be a matrix of instantaneous heart rates or a data matrix structured with two elements such as time-on-the-clock information of the first R-wave structuring an instantaneous heart rate and the instantaneous heart rate. This function is not a requisite function in the embodiment. This function is necessary only at the time of evaluating the instantaneous heart rates while also taking time information of the instantaneous heart rates into consideration in addition to the electrical potential information of the R-waves. Further, in FIG. 4, non-requisite functions are indicated as blocks outlined with broken lines so that those functions are easily identified.

On the basis of the information from the R-wave relevance information record unit 13, the instantaneous heart rate evaluation unit 16 evaluates the instantaneous heart rates calculated by the instantaneous heart rate calculation unit 14. This process is a requisite process when the instantaneous heart rate abnormality value processing unit 17 (explained later) eliminates abnormality values from instantaneous heart rates on the basis of a measurement state and time information of the instantaneous heart rates, as proposed in Non-Patent Literature 3. This process is not a requisite process when abnormality values are eliminated from the instantaneous heart rates on the basis of time information of the instantaneous heart rates as proposed in Non-Patent Literatures 1 and 2. In the embodiment, specifics of the processes performed by the instantaneous heart rate evaluation unit 16 are not defined. For example, the instantaneous heart rate evaluation unit 16 may evaluate the instantaneous heart rates as proposed in Non-Patent Literature 3.

The instantaneous heart rate abnormality value processing unit 17 eliminates abnormality values from the instantaneous heart rates, in accordance with an evaluation result from one of the instantaneous heart rate record unit 15 and the instantaneous heart rate evaluation unit 16. In the present embodiment, specifics of the processes performed by the instantaneous heart rate abnormality value processing unit 17 are not defined. Although one example of the process performed by the instantaneous heart rate abnormality value processing unit 17 will be described in the section “An operation” below, the method implemented in the process performed by the instantaneous heart rate abnormality value processing unit 17 is not limited to the method described below. For example, the instantaneous heart rate abnormality value processing unit 17 may perform only an abnormality value elimination process based on a time feature of the instantaneous heart rates only as proposed in Non-Patent Literatures 1 and 2. Alternatively, the instantaneous heart rate abnormality value processing unit 17 may perform an abnormality value elimination process based on both a measurement state and a time feature of the instantaneous heart rates as proposed in Non-Patent Literature 3. In another example, the instantaneous heart rate abnormality value processing unit 17 may perform an abnormality value elimination process based on only a measurement state of the instantaneous heart rates, without performing an abnormality value elimination based on a time feature of the instantaneous heart rates. Further, when the processes by neither the instantaneous heart rate record unit 15 nor the instantaneous heart rate evaluation unit 16 are performed, the processes by the instantaneous heart rate abnormality value processing unit 17 do not need to be performed.

The time feature value calculation processing unit 18 calculates a time feature value by using output data of the instantaneous heart rate abnormality value processing unit 17. The time feature value calculation processing unit 18 performs various calculation processes in accordance with characteristics of the time feature value to be calculated (the calculation target). In other words, the time feature value calculation processing unit 18 includes: a calculation target determination unit 181 that determines characteristics of the time feature value to be calculated (the calculation target); an instantaneous heart rate interpolation unit 182 and a first time feature value calculation unit 183 for calculating a time feature value focusing on a variability tendency of the instantaneous heart rates measured over an arbitrary time period; and a second time feature value calculation unit 184 for calculating a time feature value focusing on a relationship between adjacent instantaneous heart rates.

The calculation target determination unit 181 determines whether the characteristics of the time feature value to be calculated having been selected by the user correspond to a time feature value focusing on a variability tendency of the instantaneous heart rates measured over the arbitrary time period or to a time feature value focusing on a relationship between adjacent instantaneous heart rates and further outputs, depending on the determined result, the instantaneous heart rate time-series data resulting from the abnormality value elimination process performed by the instantaneous heart rate abnormality value processing unit 17 to one selected from between the instantaneous heart rate interpolation unit 182 and the second time feature value calculation unit 184. The user is able to select what type of time feature value is to be calculated (a calculation target), by performing an operation on the display device with touch panel 9 or on the input apparatus 3, for example. The calculation target may be selected by receiving a user selection instruction transmitted via the network NW or may be selected in advance in a setting file or the like.

The instantaneous heart rate interpolation unit 182 performs an interpolation process corresponding to the time feature value to be calculated, on the instantaneous heart rate time-series data resulting from the abnormality value elimination process performed by the instantaneous heart rate abnormality value processing unit 17 and being output from the calculation target determination unit 181. Alternatively, in the situation where the processes by the instantaneous heart rate abnormality value processing unit 17 are not performed, the instantaneous heart rate interpolation unit 182 performs the process on the instantaneous heart rate time-series data resulting from the process performed by the instantaneous heart rate calculation unit 14. A specific method will be explained later.

The first time feature value calculation unit 183 calculates the time feature value focusing on the variability tendency of the instantaneous heart rates measured over the arbitrary time period, by using the instantaneous heart rate time-series data resulting from the abnormality value elimination process performed by the instantaneous heart rate abnormality value processing unit 17 and the subsequent interpolation process performed by the instantaneous heart rate interpolation unit 182. A specific method will be explained later.

The second time feature value calculation unit 184 calculates the time feature value focusing on the relationship between adjacent instantaneous heart rates, by using the instantaneous heart rate time-series data resulting from the abnormality value elimination process performed by the instantaneous heart rate abnormality value processing unit 17 and being output from the calculation target determination unit 181. A specific method will be explained later.

The time feature value output unit 19 outputs the time feature value calculated by the time feature value calculation processing unit 18. The time feature value output unit 19 causes the calculated time feature value to be displayed by, for example, one of the display device with touch panel 9 and the input apparatus 3. Further, the time feature value output unit 19 is also capable of causing the calculated time feature value to be printed by the printing apparatus 5 or to be transmitted to an apparatus or a server in the network NW. In other examples, the time feature value output unit 19 may cause the calculated time feature value to be stored in a storage medium (not shown) or to be forwarded to another apparatus (not shown).

[Operation]

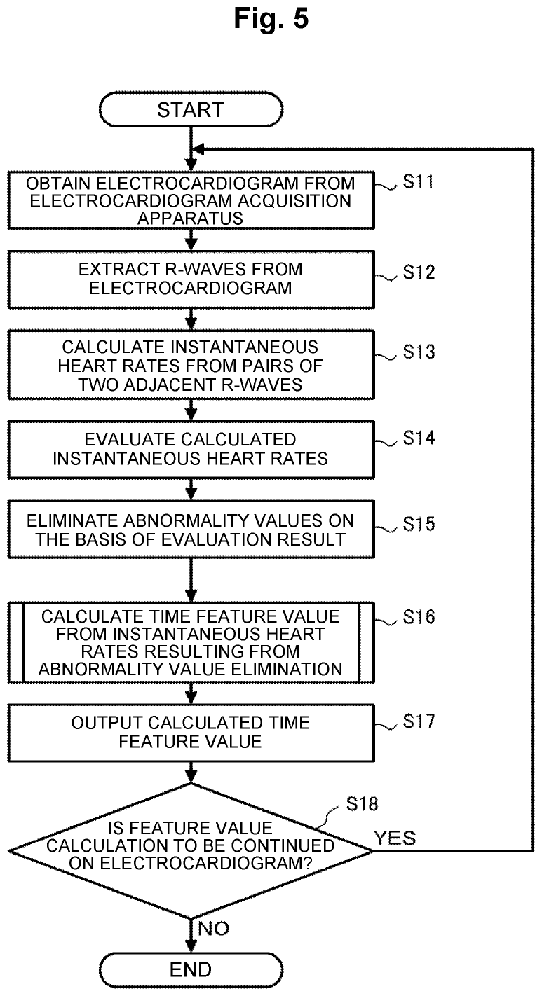

Next, an operation of the instantaneous heart rate time feature value calculation apparatus 2 according to the embodiment will be explained with reference to the flowchart in FIG. 5. The embodiment is based on the situation where only the instantaneous heart rate evaluation based on the method described in Non-Patent Literature 3 is performed, i.e., the “R-wave relevance information record unit 13” and the “instantaneous heart rate record unit 15” are provided, so that the processes of both the “instantaneous heart rate evaluation unit 16” and the “instantaneous heart rate abnormality value processing unit 17” are performed.

To begin with, the electrocardiogram obtainment unit 11 obtains the electrocardiogram acquired by the electrocardiogram acquisition apparatus 1 and sends the obtained electrocardiogram to the R-wave extraction unit (step S11).

The R-wave extraction unit 12 extracts R-waves from the electrocardiogram acquired by the electrocardiogram acquisition apparatus 1 (step S12). In the present embodiment, the instantaneous heart rate evaluation unit 16 makes an evaluation by using the measurement state and the time information of the instantaneous heart rates, by implementing a method such as that proposed in Non-Patent Literature 3. Accordingly, the R-wave extraction unit 12 records the information related to the calculated R-waves into the R-wave relevance information record unit 13. When the measurement state of the instantaneous heart rates is not to be used in the abnormality value elimination process, the R-wave extraction unit 12 does not necessarily have to record the information related to the calculated R-waves into the R-wave relevance information record unit 13.

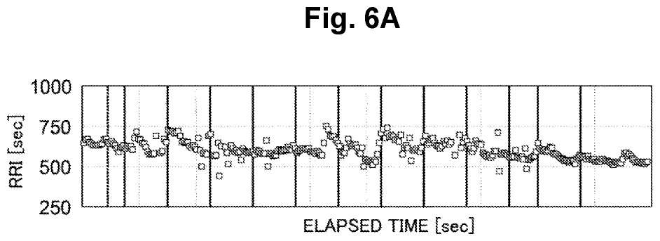

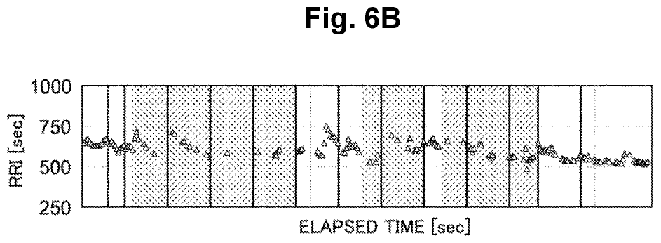

The instantaneous heart rate calculation unit 14 calculates an instantaneous heart rate from each pair of two adjacent R-waves, on the basis of the R-waves obtained by the R-wave extraction unit 12 (step S13). In the present embodiment, the instantaneous heart rate evaluation unit 16 makes the evaluation by using the measurement state and the time information of the instantaneous heart rates, by implementing a method such as that proposed in Non-Patent Literature 3. For this reason, the instantaneous heart rate calculation unit 14 records the information related to the calculated instantaneous heart rates into the instantaneous heart rate record unit 15. In the situation where the time information of the instantaneous heart rates is not used in the abnormality value elimination process, the instantaneous heart rate calculation unit 14 does not have to record the information related to the calculated instantaneous heart rates into the instantaneous heart rate record unit 15. FIG. 6A is a chart showing an example of the time-series data of the instantaneous heart rates, which serves as an example of the information related to the calculated instantaneous heart rates.

The instantaneous heart rate evaluation unit 16 evaluates the instantaneous heart rates, on the basis of the information about the R-waves recorded in the R-wave relevance information record unit 13 and the information related to the instantaneous heart rates either being calculated by the instantaneous heart rate calculation unit 14 or being recorded in the instantaneous heart rate record unit 15 (step S14). In the present embodiment, the specific evaluation method is not particularly defined. For example, possible evaluation methods include one based on measurement states of the two R-waves structuring an instantaneous heart rate, such as the method proposed in Non-Patent Literature 3.

In the present method, the measurement state of an instantaneous heart rate is evaluated on the basis of measurement states of the two R-waves structuring the instantaneous heart rate. According to the present method, the instantaneous heart rate evaluation unit 16 assigns evaluation values corresponding to combinations of measurement states of the R-waves structuring an instantaneous heart rate, as shown in Table 1 presented below.

TABLE 1

| | Determination | | Evaluation |
|---|---|---|---|
| # | Results | Details of the State | Values |
| 1 | “R” and “R” | Both are in the normal | 1 |
| | | measurement state | |
| 2 | “R” and “A” | One is in the normal measurement | 0.4 |
| 3 | “A” and “R” | state, and the other has an artifact | |
| 4 | “A” and “A” | Both have an artifact | 0 |

In other words, as examples of the R-wave measurement state, when two types of state, namely a normal measurement state and an artifact, are considered, the combination in a result of determining the measurement states of the R-waves structuring an instantaneous heart rate obtained by the instantaneous heart rate evaluation unit 16 is one of the patterns indicated with serial numbers #1 to #4 in Table 1. In this situation, the format of the serial numbers is not limited to the above. In the determination results in Table 1, “R” denotes the normal measurement state, whereas “A” denotes an artifact. In other words, the determination result “R and R” corresponding to serial number #1 in Table 1 indicates that the determination results of the measurement states of both the first and the second R-waves that are adjacent to each other are each the normal measurement state. The determination result “R and A” corresponding to serial number #2 in Table 1 indicates that the determination result of the measurement state of the first R-wave of two adjacent R-waves is the normal state, whereas the determination result of the measurement state of the second R-wave is an artifact. The determination result “A and R” corresponding to serial number #3 in Table 1 indicates that the determination result of the measurement state of the first R-wave of two adjacent R-waves is an artifact, whereas the determination result of the measurement state of the second R-wave is the normal state. The determination result “A and A” corresponding to serial number #4 in Table 1 indicates that the determination results of the measurement states of both the first and the second R-waves that are adjacent to each other are each an artifact.

In Table 1, the section “Details of the State” describes details of the measurement states of the two adjacent R-waves, based on the determination result shown in the same line of Table 1.

In the expressions used in the section “Details of the State”, only the combinations in the determination results of the measurement states of the two R-waves structuring an instantaneous heart rate are distinguished, and whether the R-wave is the earlier one or the later one in the time sequence is not distinguished. In other words, although the example in Table 1 shows four combinations corresponding to serial numbers #1 to #4 as the combinations of the determination results of the measurement states, “Details of the State” corresponding to serial numbers #2 and #3 both equally denote “One is in the normal measurement state, while the other has an artifact”. Accordingly, “Details of the State” in Table 1 show three possibilities.

In the present embodiment, an example will be explained in which an evaluation is made for each item of “Details of the State” in Table 1. However, it is also acceptable to provide other evaluation criteria. For example, for the same description under the “Details of the State”, it is acceptable to provide an evaluation criterion that distinguishes the earlier R-wave from the later R-wave between the two R-waves.

The instantaneous heart rate evaluation unit 16 assigs one of mutually-different evaluation values to each of the states, so that the details of the state are easily distinguished from one another. Examples of the evaluation values are indicated under “Evaluation Values” in Table 1. These evaluation values are merely examples and the method for determining the evaluation values is not particularly limited in the present embodiment. Next, the “Evaluation Values” in Table 1 will be explained. The “Evaluation Values” each express reliability of the measurement state by using a numerical value between 0 and 1 inclusive, with respect to each of the two R-waves structuring an instantaneous heart rate, the measurement state being indicated under the “Details of the State” in the same line of Table 1, so as to assign an arbitrary evaluation value to each of the states listed under the “Details of the State”. The ranges of the evaluation values and the increments of the evaluation values corresponding to the various states are not particularly limited. For example, evaluation values between 1 and 10, inclusive, may be assigned to the various states with increments of 1. In another example, the increments of the evaluation values may be different among the various states. In yet another example, the lengths of bars in a horizontal bar graph, for instance, may be used in place of the evaluation values (e.g., the longer the length of a horizontal bar in the graph is, the higher is the reliability).

Specific examples of the evaluation values will be explained. As shown in Table 1, when “both are in the normal measurement state” under the “Details of the State” identified with serial number #1, the “Evaluation Value” for serial number #1 is “1”, which is the largest value.

Further, similarly to the expression used in the “Details of the State”, the expressions used in the “Evaluation Values” distinguish only the combinations of the determination results of the measurement states of the two R-waves structuring an instantaneous heart rate, and do not distinguish the earlier R-wave from the later R-wave in the time sequence. In other words, the “Details of the State” identified with serial numbers #2 and #3 both equally denote “One is in the normal measurement state, while the other has an artifact”. Thus, the “Evaluation Values” for serial numbers #2 and #3 are both “0.4”, which is smaller than the “Evaluation Value” for serial number #1 by 0.6.

The “Details of the State” identified with serial number #4 indicate “Both have an artifact”, and the “Evaluation Value” for serial number #4 is “0”, which is the smallest value and is smaller than the “Evaluation Value” for #2 and #3 by 0.4.

On the basis of the evaluation results obtained by the instantaneous heart rate evaluation unit 16, the instantaneous heart rate abnormality value processing unit 17 regards any instantaneous heart rate having a smaller value than an evaluation value set for an abnormality value determination purpose as an abnormality value so as to eliminate such instantaneous heart rates from the instantaneous heart rate time-series data to be forwarded to the subsequent process (step S15). In the present embodiment, the specific elimination method is not particularly defined. Examples of the method include the one proposed in Non-Patent Literature 3. In this method, for example, when no artifact should be contained in the instantaneous heart rate time-series data to be forwarded to the subsequent process, the evaluation value needs to be “1”. Accordingly, the instantaneous heart rate abnormality value processing unit 17 regards any instantaneous heart rate having an evaluation value equal to or smaller than “0.4” as an abnormality value and eliminates such instantaneous heart rates from the instantaneous heart rate time-series data to be forwarded to the subsequent process. After that, the instantaneous heart rate abnormality value processing unit 17 eliminates instantaneous heart rates lower than 250 [msec] and higher than 1,500 [msec] from the instantaneous heart rate time-series data to be forwarded to the subsequent process and also eliminates any instantaneous heart rate outside the range of “An average ±3× the standard deviation” as an abnormality value.

FIG. 6B is a chart showing an example of a result of the instantaneous heart rate abnormality value processing unit 17 eliminating abnormality values from instantaneous heart rates, with respect to the time-series data of the instantaneous heart rate shown in FIG. 6A. The number of instantaneous heart rates in the section indicated with the hatching where many measurement abnormalities occurred is significantly different from the number of the other instantaneous heart rates. It is therefore difficult to understand the variability tendency of the instantaneous heart rates in the hatching section.

The time feature value calculation processing unit 18 calculates a time feature value from the instantaneous heart rate time-series data from which the abnormality values have been eliminated by the instantaneous heart rate abnormality value processing unit 17 (step S16). The calculation process varies depending on the characteristics of the time feature value being calculated (the calculation target). More specifically, mutually-different processes are performed for the following situations: the situation where the calculation target includes a time feature value focusing on a relationship between adjacent instantaneous heart rates (hereinafter, “time feature value based on an adjacent characteristic of the instantaneous heart rates”); and the situation where the calculation target includes a time feature value focusing on a variability tendency of instantaneous heart rates measured over an arbitrary time period (hereinafter, “time feature value reflecting a variability tendency of the instantaneous heart rates”). When the two types of time feature values are both calculation targets, the processes described below are each performed appropriately, but the order of execution is not particularly defined in the present embodiment.

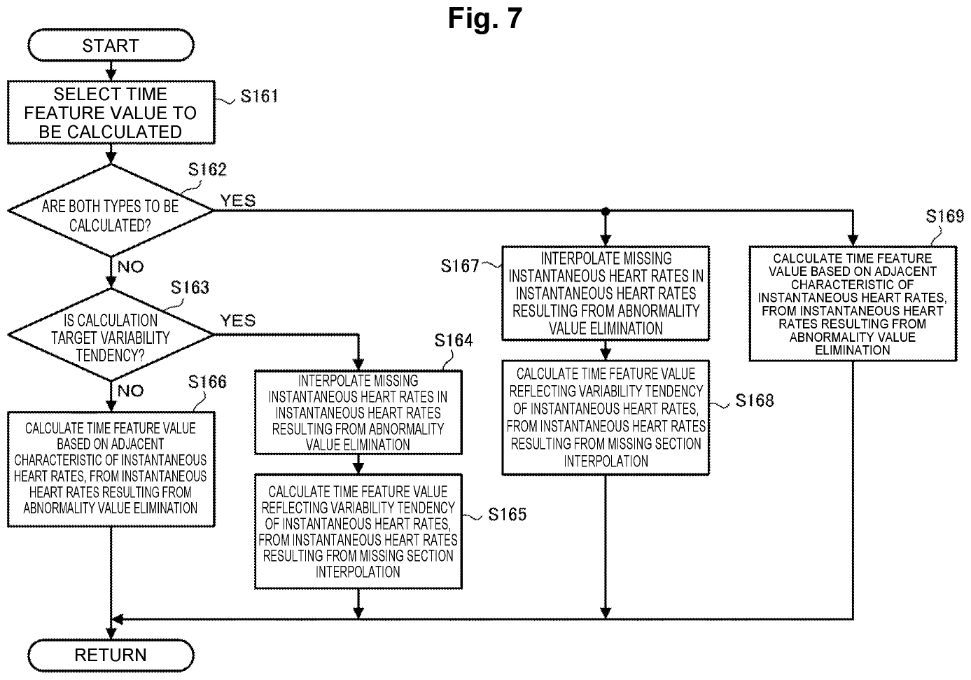

FIG. 7 is a flowchart for explaining the process at step S16 of calculating the time feature value from the instantaneous heart rate time-series data from which the abnormality values have been eliminated.

As shown in FIG. 7, the time feature value calculation processing unit 18 at first selects a time feature value to be calculated (a calculation target) (step S161). In this situation, for example, selection candidates for the time feature value to be calculated may include: the square root of the mean square value of temporal differences between adjacent RRIs (RMSSD); the standard deviation of temporal differences between adjacent normal RRIs (SDSD); a total number of pairs of adjacent RRIs between which the temporal difference exceeds 50 milliseconds (NN50); the percentage of the number of times of occurrence where the temporal difference between a pair of adjacent RRIs is equal to or longer than 50 milliseconds (pNN50); a parasympathetic nerve activity index calculated from the length L of the long side and the length T of the short side of a shape drawn by a Lorenz plot of adjacent RRIs (CVI); a sympathetic nerve activity index calculated from L and T (CSI); an average heart rate; an average RRI, a variability coefficient of RRIs (CVNN); the standard deviation of RRIs (SDNN); and the standard deviation of an average RRI over a prescribed time period (SDANN). Needless to say, there may be other selection candidates. RMSSD, SDSD, NN50, pNN50, CVI, and CSI are time feature values based on an adjacent characteristic of the instantaneous heart rates. An average heart rate, an average RRI, CVNN, SDNN, and SDANN are time feature values reflecting a variability tendency of the instantaneous heart rates. The time feature value to be calculated may be selected according to a user operation performed on the display device with touch panel 9 or on the input apparatus 3, or may be selected according to a setting made in advance using a file or a variable.

The time feature value calculation processing unit 18 determines whether or not the characteristics of the time feature value selected by the calculation target determination unit 181 as the calculation target correspond to both a time feature value based on an adjacent characteristic and a time feature value based on a variability tendency (step S162). When the calculation target determination unit 181 determines that the characteristics of the time feature value selected as the calculation target do not correspond to both, but correspond to one of the two, the time feature value calculation processing unit 18 further determines whether or not the characteristics of the time feature value selected by the calculation target determination unit 181 as the calculation target correspond to a time feature value based on a variability tendency (step S163).

When a time feature value reflecting a variability tendency of the instantaneous heart rates (e.g., an average heart rate, an average RRI, CVNN, SDNN, or SDANN) is selected as the calculation target, the calculation target determination unit 181 determines that the characteristics of the time feature value selected as the calculation target correspond to a time feature value based on a variability tendency. In that situation, the time feature value calculation processing unit 18 causes an instantaneous heart rate interpolation unit 182 to perform an interpolation process on the instantaneous heart rate time-series data having missing values as a result of the process performed by the instantaneous heart rate abnormality value processing unit 17 (step S164). The feature values described above are merely examples, and the processes are not limited to these feature values. As long as the time feature value focuses on a variability tendency of heart rates over a prescribed time period, the calculation target determination unit 181 may determine that the calculation target is a time feature value reflecting a variability tendency of the instantaneous heart rates and cause the instantaneous heart rate interpolation unit 182 to perform the interpolation process.

As for the interpolation process performed by the instantaneous heart rate interpolation unit 182, the present embodiment does not designate a specific mathematical function; however, it is possible to use, for example, any of the following mathematical functions each corresponding to a variability tendency of the instantaneous heart rates:

(1) A linear interpolation based on the values before and after a missing value

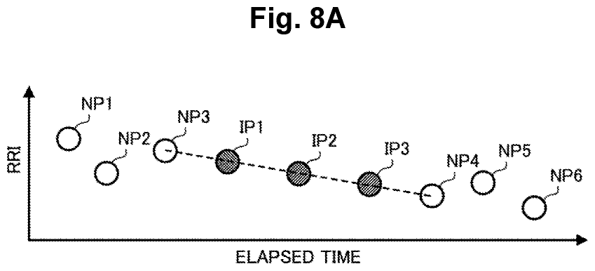

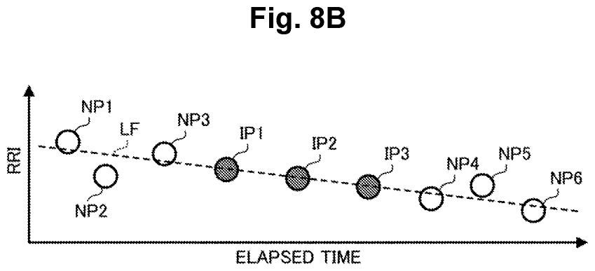

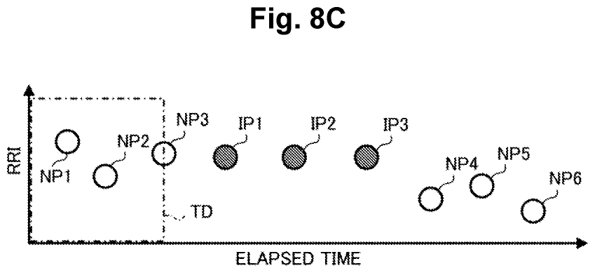

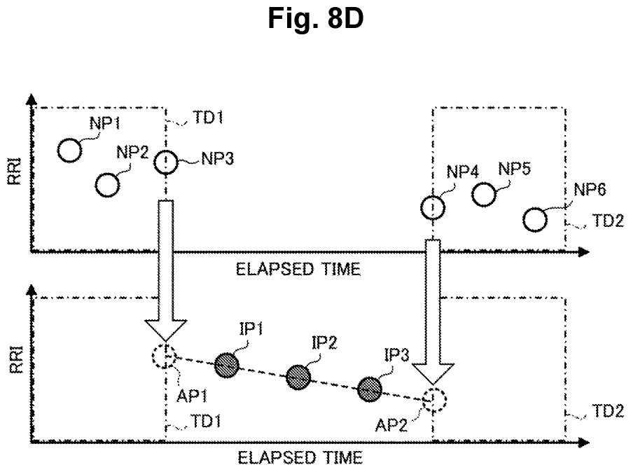

This type of interpolation will be explained by using an example of instantaneous heart rate time-series data including instantaneous heart rates NP1, NP2, NP3, NP4, NP5, and NP6 as shown in FIG. 8A which are among a plurality of instantaneous heart rates determined as normal values and are normally measured over a prescribed time period. In this situation, because instantaneous heart rate missing values are between the instantaneous heart rates NP3 and NP4, the instantaneous heart rate interpolation unit 182 calculates instantaneous heart rates IP1, IP2, and IP3, by performing a linear interpolation based on the value of the instantaneous heart rate NP3 and the value of the instantaneous heart rate NP4.

In this situation, the specific intervals of the interpolation are not particularly defined. The same applies to the other interpolation processes described below. It is acceptable to complement the values discretely and at irregular intervals by using only such values that are considered reasonable in an RRI tacogram. Alternatively, it is also acceptable to complement the values continuously and at regular intervals according to an arbitrary sampling rate.

(2) An interpolation using a linear function or the like approximating an RRI variability tendency measured over a prescribed time period

For example, as shown in FIG. 8B, the instantaneous heart rate interpolation unit 182 calculates a linear function LF approximating a variability tendency of the instantaneous heart rates NP1, NP2, NP3, NP4, NP5, and NP6 normally measured over the prescribed time period and further calculates the instantaneous heart rates IP1, IP2, an IP3 which are interpolation values, by using the linear function LF.

(3) An interpolation using an average value of pieces of data sectioned by an arbitrary duration.

For example, as shown in FIG. 8C, the instantaneous heart rate interpolation unit 182 calculates an average of the values of the instantaneous heart rates NP1, NP2, and NP3 in a data section TD sectioned by an arbitrary duration and adopts the average value as the values of the instantaneous heart rates IP1, IP2, and IP3.

(4) A linear interpolation based on an average value of data sections each sectioned by an arbitrary duration

In this method, an average value of data sections is calculated, so as to perform a linear interpolation on the basis of two average values. For example, as shown in FIG. 8D, the instantaneous heart rate interpolation unit 182 calculates an average AP1 of the values of the instantaneous heart rates NP1, NP2, and NP3 in the data section TD1 sectioned by an arbitrary duration and also calculates an average AP2 of the values of the instantaneous heart rates NP4, NP5, and NP6 in another data section TD2 similarly sectioned by the arbitrary duration. Further, the instantaneous heart rate interpolation unit 182 calculates the instantaneous heart rates IP1, IP2, and IP3, by performing a linear interpolation based on the value of the average AP1 and the value of the average AP2. Although the data sections TD1 and TD2 have mutually the same duration in the present example, the data sections TD1 and TD2 may be different from each other.

The mathematical functions (1) to (4) are merely examples, and possible processes are not limited to these mathematical functions. As long as an expression is able to reflect a variability tendency of the instantaneous heart rates, the expression can serve as a candidate for the mathematical function used in the interpolation.

Subsequently, by using the instantaneous heart rate time-series data resulting from the missing section interpolation process performed by the instantaneous heart rate interpolation unit 182, the time feature value calculation processing unit 18 causes the first time feature value calculation unit 183 to calculate a time feature value reflecting a variability tendency of the instantaneous heart rates (step S165). In other words, the first time feature value calculation unit 183 calculates a selected time feature value such as an average heart rate, an average RRI, CVNN, SDNN, or SDANN. When the time feature value has been calculated in the manner, the instantaneous heart rate time feature value calculation apparatus 2 proceeds to the subsequent process at step S17.

Even when the interpolation process is performed in the manner described above, the first time feature value calculation unit 183 may be configured to also calculate a time feature value using instantaneous heart rate time-series data that did not undergo any interpolation process, as reference information for making clear the variability of the heart rate feature value resulting from the interpolation process.

In contrast, at step S163, when the calculation target includes a time feature value based on an adjacent characteristic of the instantaneous heart rates such as RMSSD, SDSD, NN50, pNN50, CVI, or CSI, the calculation target determination unit 181 determines that the characteristics of the time feature value selected as the calculation target do not correspond to a time feature value based on a variability tendency. In that situation, the time feature value calculation processing unit 18 causes the second time feature value calculation unit 184 to calculate a time feature value based on an adjacent characteristic of the instantaneous heart rates, from the instantaneous heart rate time-series data resulting from the abnormality value process performed by the instantaneous heart rate abnormality value processing unit 17 (step S166). In other words, the second time feature value calculation unit 184 calculates the selected time feature value such as RMSSD, SDSD, NN50, pNN50, CVI, or CSI. When the time feature value has been calculated in this manner, the instantaneous heart rate time feature value calculation apparatus 2 proceeds to the subsequent process at step S17.

Further, let us discuss the situation where, at step S162, the calculation target determination unit 181 determines that the selected calculation target corresponds to both a time feature value based on an adjacent characteristic and a time feature value based on a variability tendency. In this situation, the time feature value calculation processing unit 18 causes the instantaneous heart rate interpolation unit 182 and the first time feature value calculation unit 183 to perform the same processes as those at steps S164 and S165 described above and further causes the second time feature value calculation unit 184 to perform the same process as that at step S166 described above. In other words, the time feature value calculation processing unit 18 causes the instantaneous heart rate interpolation unit 182 to perform the interpolation process on the instantaneous heart rate time-series data having the missing values as a result of the process performed by the instantaneous heart rate abnormality value processing unit 17 (step S167). Subsequently, the time feature value calculation processing unit 18 causes the first time feature value calculation unit 183 to calculate a time feature value reflecting a variability tendency of the instantaneous heart rates, by using the instantaneous heart rate time-series data resulting from the missing section interpolation process performed by the instantaneous heart rate interpolation unit 182 (step S168). Further, in parallel to the processes at steps S167 and S168, the time feature value calculation processing unit 18 causes the second time feature value calculation unit 184 to calculate a time feature value based on an adjacent characteristic of the instantaneous heart rates, from the instantaneous heart rate time-series data having the missing values as a result of the process performed by the instantaneous heart rate abnormality value processing unit 17 (step S169). When the two types of time feature values have been calculated in this manner, the instantaneous heart rate time feature value calculation apparatus 2 proceeds to the subsequent process at step S17. Alternatively, the processes above may be performed as successive processes where the processes at steps 5167 and 5168 are performed following the process at step S169.

When the time feature value of the instantaneous heart rates has been calculated by the time feature value calculation processing unit 18 in this manner, the instantaneous heart rate time feature value calculation apparatus 2 causes the time feature value output unit 19 to output the calculated time feature value of the instantaneous heart rates (step S17). For example, the time feature value output unit 19 may cause the calculated time feature value to be displayed by one of the display device with touch panel 9 and the input apparatus 3, to be stored in a storage medium (not shown), or to be forwarded to an external apparatus.

After that, in response to a user operation performed on the display device with touch panel 9 or the input apparatus 3, the instantaneous heart rate time feature value calculation apparatus 2 determines whether or not there is an instruction to continue calculating a feature value from the electrocardiogram (step S18). In other words, the instantaneous heart rate time feature value calculation apparatus 2 determines whether or not there is an instruction to process the part later than the part of the electrocardiogram from which the time feature value has been calculated. When having determined that there is such an instruction, the instantaneous heart rate time feature value calculation apparatus 2 returns to the process at step Sll described above.

On the contrary, when having determined that there is no such instruction, the instantaneous heart rate time feature value calculation apparatus 2 ends the series of processes shown in FIG. 5.

As explained above, by performing the missing value interpolation before calculating the feature value, the instantaneous heart rate time feature value calculation apparatus 2 according to the embodiment of the present invention is able to calculate, with excellent precision, the time feature value focusing on the variability tendency of the instantaneous heart rates measured over the arbitrary time period, from the instantaneous heart rate time-series data having the missing section caused by a measurement abnormality or the like.

In other words, in the instantaneous heart rate time feature value calculation apparatus 2 according to the embodiment of the present invention, the instantaneous heart rate interpolation unit 182 interpolates the instantaneous heart rate missing values in the instantaneous heart rate time-series data having the missing values, in accordance with the variability tendency of the instantaneous heart rates exhibited in the instantaneous heart rate time-series data resulting from the instantaneous heart rate abnormality value process performed by the instantaneous heart rate abnormality value processing unit 17 to eliminate the instantaneous heart rate abnormality values in response to the input of the instantaneous heart rates. Further, the first time feature value calculation unit 183 calculates the time feature value focusing on the variability tendency of the instantaneous heart rates measured over the arbitrary time period, from the instantaneous heart rate time-series data resulting from the interpolation. With this arrangement, it is possible to calculate, with excellent precision, the time feature value focusing on the variability tendency of the instantaneous heart rates measured over the arbitrary time period, from the instantaneous heart rate time-series data having the missing section caused by a measurement abnormality or the like.

Further, in the instantaneous heart rate time feature value calculation apparatus 2 according to the embodiment of the present invention, the instantaneous heart rate interpolation unit 182 is able to interpolate the missing values in the instantaneous heart rates while reflecting the variability tendency of the instantaneous heart rates, by interpolating the instantaneous heart rate missing values while performing one of the following: a linear interpolation; an interpolation using a mathematical function approximating the instantaneous heart rate variability tendency; and an interpolation using an average value of the data sectioned by the arbitrary duration.

Further, the instantaneous heart rate time feature value calculation apparatus 2 according to the embodiment of the present invention further includes the calculation target determination unit 181 that determines whether the characteristics of the time feature value to be calculated (the calculation target) correspond to a time feature value focusing on a variability tendency of the instantaneous heart rates measured over the arbitrary time period or to a time feature value focusing on a relationship between adjacent instantaneous heart rates. Further, the instantaneous heart rate interpolation unit 182 operates only when the calculation target determination unit 181 determines that the characteristics of the time feature value being the calculation target correspond to a time feature value focusing on a variability tendency of the instantaneous heart rates measured over the arbitrary time period. With this arrangement, it is possible to cause the instantaneous heart rate interpolation unit 182 and the first time feature value calculation unit 183 to operate only when the calculation target is a time feature value focusing on a variability tendency of the instantaneous heart rates measured over the arbitrary time period.

### MODIFICATION EXAMPLES

In the embodiment above, in the interpolation process performed by the instantaneous heart rate interpolation unit 182 at step S164 or step S167 is performed also when one of the time feature values reflecting a variability tendency of the instantaneous heart rates such as an average heart rate, an average RRI, CVNN, SDNN, or SDANN is selected as the calculation target. However, depending on the type of the selected time feature value, the interpolation process does not necessarily have to be performed. For example, an arrangement is acceptable in which the interpolation is performed only when one of an average RRI and an average heart rate is selected, which are each a time feature value focusing on a variability tendency of the instantaneous heart rates in a broader perspective, whereas the interpolation is not performed when one of SDNN and SDANN is selected, each of which focuses on a variability tendency in a narrower perspective than the above time feature values.

In that situation, the calculation target determination unit 181 may have the function to determine the type of the time feature value and to output the instantaneous heart rate time-series data resulting from the abnormality value elimination process performed by the instantaneous heart rate abnormality value processing unit 17, to one selected from between the instantaneous heart rate interpolation unit 182 and the first time feature value calculation unit 183, depending on the determination result. In other words, when the type of the time feature value selected as the calculation target is one of an average RRI and an average heart rate, the calculation target determination unit 181 outputs the instantaneous heart rate time-series data resulting from the abnormality value elimination process to the instantaneous heart rate interpolation unit 182. In contrast, when the type of the time feature value selected as the calculation target is one of SDNN and SDANN, the calculation target determination unit 181 outputs the instantaneous heart rate time-series data resulting from the abnormality value elimination process to the first time feature value calculation unit 183.

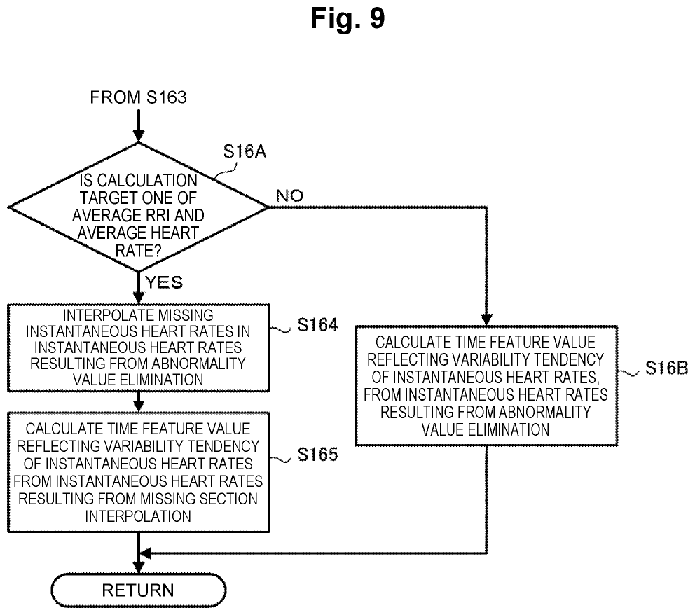

FIG. 9 is a flowchart for explaining a modified part of the process at step S16 according to the present modification example. As shown in FIG. 9, the processes at steps S16A and S16B are added to the flowchart shown in FIG. 7. In other words, when the calculation target determination unit 181 determines at step S163 that the characteristics of the time feature value selected as the calculation target correspond to a time feature value based on a variability tendency, the time feature value calculation processing unit 18 further causes the calculation target determination unit 181 to determine whether or not the type of the time feature value selected as the calculation target is one of an average RRI and an average heart rate (step S16A). In this situation, when the calculation target determination unit 181 determines that the type of the time feature value selected as the calculation target is one of an average RRI and an average heart rate, the time feature value calculation processing unit 18 causes the instantaneous heart rate interpolation unit 182 and the first time feature value calculation unit 183 to perform the processes at steps S164 and S165 described above.

Let us discuss the situation where, on the contrary, the calculation target determination unit 181 determines at step S16A above that the type of the time feature value selected as the calculation target is not one of an average RRI and an average heart rate. In that situation, the time feature value calculation processing unit 18 causes the first time feature value calculation unit 183 to calculate a time feature value reflecting a variability tendency of the instantaneous heart rates, by using the instantaneous heart rate time-series data having missing values as a result of the process performed by the instantaneous heart rate abnormality value processing unit 17 (step S16B). In other words, the first time feature value calculation unit 183 calculates a selected time feature value such as SDNN or SDANN. When the time feature value has been calculated in this manner, the instantaneous heart rate time feature value calculation apparatus 2 proceeds to the subsequent process at step S17.

As explained above, it is possible to determine whether or not the interpolation process is to be performed thereon, depending on the type of the time feature value in addition to the characteristics of the time feature value.

Alternatively, when the calculation target determination unit 181 determines that the type of the time feature value selected as the calculation target is not one of an average RRI and an average heart rate, the instantaneous heart rate time-series data resulting from the abnormality value elimination process may be output to the instantaneous heart rate interpolation unit 182 together with the determination result, instead of being output to the first time feature value calculation unit 183. In this situation, the instantaneous heart rate interpolation unit 182 may be configured to determine whether or not the interpolation process is to be performed on the instantaneous heart rate time-series data resulting from the abnormality value elimination process, depending on the determination result of the calculation target determination unit 181. In other words, when the determination result from the calculation target determination unit 181 indicates that the type of the time feature value selected as the calculation target is one of an average RRI and an average heart rate, the instantaneous heart rate interpolation unit 182 performs the interpolation process on the received time-series data of the instantaneous heart rates resulting from the abnormality value elimination process and subsequently outputs the result thereof to the first time feature value calculation unit 183. On the contrary, when the determination result from the calculation target determination unit 181 indicates that the type of the time feature value selected as the calculation target is not one of an average RRI and an average heart rate, the instantaneous heart rate interpolation unit 182 does not perform the interpolation process, but outputs the received time-series data of the instantaneous heart rates resulting from the abnormality value elimination process to the first time feature value calculation unit 183 without performing any process thereon.

As explained above, in the instantaneous heart rate time feature value calculation apparatus 2 according to the modification example of the embodiment of the present invention, the calculation target determination unit 181 determines whether or not the interpolation by the instantaneous heart rate interpolation unit 182 is to be performed, depending on the type of the time feature value which is to be calculated by the first time feature value calculation unit 183 and which focuses on a variability tendency of the instantaneous heart rates measured over the arbitrary time period. When the calculation target determination unit 181 determines that the interpolation by the instantaneous heart rate interpolation unit 182 is not to be performed, the first time feature value calculation unit 183 calculates the time feature value focusing on the variability tendency of the instantaneous heart rates measured over the arbitrary time period, from the instantaneous heart rate time-series data resulting from the abnormality value elimination. In other words, even when a time feature value focusing on a variability tendency of the instantaneous heart rates measured over the arbitrary time period is selected as a calculation target, the interpolation process is performed only when the selected time feature value focuses on a variability tendency of the instantaneous heart rates in a broad perspective to a certain extent, whereas the interpolation process is not performed when the selected time feature value focuses on a variability tendency in a narrower perspective than the broad-perspective time feature values. In this situation, the type of time feature value focusing on a variability tendency of the instantaneous heart rates in a broad perspective to a certain extent denotes one of an average RRI and an average heart rate. The type of time feature value focusing on a variability tendency in a narrower perspective denotes one of SDNN and SDANN. As a result, when such a time feature value focusing on a variability tendency of the instantaneous heart rates measured over an arbitrary time period, which does not require the interpolation process, is selected as a calculation target, it is possible to omit the interpolation process and to calculate the time feature value quickly.

Further, in the embodiment, the time feature value to be calculated (the calculation target) is selected at step S161 described above; however, the selection may be made at an arbitrary earlier step. For example, the time feature value to be calculated may be selected when the instantaneous heart rate abnormality value processing unit 17 performs the abnormality value elimination process at step S15 described above, based on the evaluation result. In the embodiment, the instantaneous heart rate abnormality value processing unit 17 uniformly regards any instantaneous heart rate having an evaluation value equal to or smaller than the evaluation value “0.4” as an abnormality value and eliminates the abnormality value. In contrast, in the situation where the time feature value to be calculated is selected at step S15, the instantaneous heart rate abnormality value processing unit 17 is able to change the determination criterion used for eliminating the abnormality values, depending on the time feature value selected as the calculation target. For example, the instantaneous heart rate abnormality value processing unit 17 is able to eliminate abnormality values while regarding any instantaneous heart rate having an evaluation value smaller than “0.4” as an abnormality value if a time feature value reflecting a variability tendency of the instantaneous heart rates is selected and regarding any instantaneous heart rate having an evaluation value smaller than “1.0” as an abnormality value if a time feature value based on an adjacent characteristic of the instantaneous heart rates is selected.

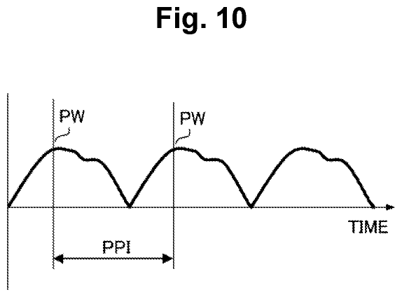

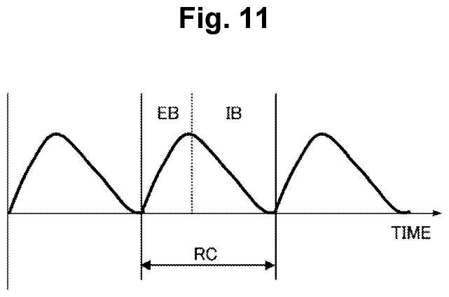

In the embodiment, the example is explained in which the instantaneous heart rates observed in the electrocardiogram are used; however, the present invention is also applicable to other biological signal processing, as long as the biological signal has arbitrary periodicity and characteristics of the biological signal are to be temporally analyzed. Examples of the biological signals include a pulse obtained as a result of analyzing a pulse wave indicated in FIG. 10 and a respiration cycle obtained as a result of analyzing the respiration indicated in FIG. 11. In FIG. 10, PPI denotes the interval between the two adjacent P-waves PW. In FIG. 11, RC denotes the respiration cycle constituted of exhalation EB and inhalation IB.

When the pulse is subject to an analysis, a “pulse wave obtainment unit”, a “P-wave extraction unit”, and a “pulse calculation unit” are provided in place of the “electrocardiogram obtainment unit 11”, the “R-wave extraction unit 12”, and the “instantaneous heart rate calculation unit 14” shown in FIG. 4. Also, a “pulse interpolation unit” is provided in place of the “instantaneous heart rate interpolation unit 182” included in the time feature value calculation processing unit 18. Further, examples of time feature values reflecting a variability tendency of the pulse and corresponding to the time feature value reflecting a variability tendency of the instantaneous heart rates used at step S165 or step S168 described above shown in FIG. 7 include: an average pulse rate, an average PPI, a variance or the standard deviation of the pulse, the square root of the mean square value of temporal differences between adjacent PPIs, and the standard deviation of temporal differences between adjacent PPIs.

Further, when a respiration interval time is subject to an analysis, a “respiration curve obtainment unit”, a “respiration feature point extraction unit”, and a “respiration cycle calculation unit” are provided in place of the “electrocardiogram obtainment unit 11”, the “R-wave extraction unit 12”, and the “instantaneous heart rate calculation unit 14” shown in FIG. 4. A “respiration cycle interpolation unit” is provided in place of the “instantaneous heart rate interpolation unit 182” included in the time feature value calculation processing unit 18. Further, examples of time feature values reflecting a variability tendency of the respiration cycle and corresponding to the time feature value reflecting a variability tendency of the instantaneous heart rates used at step S165 or step S168 described above shown in FIG. 7 include: an average respiration rate, an average respiration cycle, a variance or the standard deviation of the respiration cycle, the square root of the mean square value of temporal differences between adjacent respiration cycles, and the standard deviation of temporal differences between adjacent respiration cycles. Further, in addition to the abovementioned feature values, an average, a variance, or the standard deviation of inhalation time periods or exhalation time periods may also serve as candidates in place of the respiration cycle.

The invention of the present disclosure is not limited to the above embodiments and may be modified in various manners at the stage of being carried out, without departing from the scope thereof. Further, two or more of the embodiments may be carried out in combination, as appropriate, whenever possible. On such occasion, combined advantageous effects will be achieved. Further, the above embodiments include inventions at various stages. Accordingly, by appropriately combining two or more of the disclosed constituent elements together, it is possible to extract various inventions.

### REFERENCE DOCUMENTS

(i) Inoue, H., Junkanki Shikkan to Jiritsu Shinkei Kinou [Circulatory Diseases and Autonomic Nerve Functions (in Japanese)], second edition, Igaku Shoin, 2001
(ii) Okude, J., Korenara Wakaru! Kantan Point Shindenzu [Easy to Understand! Simple Tips for Electrocardiograms (in Japanese)], second edition, Igaku Shoin, 2011
(iii) Task Force of The European Society of Cardiology and The North American Society of Pacing and Electrophysiology, Heart rate variability: Standards of measurement, physiological interpretation, and clinical use, European Heart Journal, vol. 17, pp. 354-381, 1996.
(iv) Nihon Kohden, Zatsuon Konnyu no Mechanism to Taisaku: Kireina Shindenzu o Kiroku Suru Point—Holter Shindenzu Hen—[Mechanism of and Countermeasures for Noise Contamination: Tips for Recording Clear Electrocardiograms—for Holter Electrocardiograms—(in Japanese)] (accessed on Oct. 25, 2018). http://www.nihonkohden.co.jp/iryo/point/holter/mechanism. html

### REFERENCE SIGNS LIST

1 Electrocardiogram Acquisition Apparatus

2 Instantaneous Heart Rate Time Feature Value Calculation Apparatus

3 Input Apparatus

4 Display Apparatus

5 Printing Apparatus

6 Processor

7 Memory

8 Interface

9 Display Device with Touch Panel

11 Electrocardiogram Obtainment Unit

12 R-wave Extraction Unit

13 R-wave Relevance Information Record Unit

14 Instantaneous Heart Rate Calculation Unit

15 Instantaneous Heart Rate Record Unit

16 Instantaneous Heart Rate Evaluation Unit

17 Instantaneous Heart Rate Abnormality Value Processing Unit

18 Time Feature Value Calculation Processing Unit

19 Time Feature Value Output Unit

181 Calculation Target Determination Unit

182 Instantaneous Heart Rate Interpolation Unit

183 First Time Feature Value Calculation Unit

184 Second Time Feature Value Calculation Unit

1. A time feature value calculation apparatus comprising:
a processor; and
a storage medium having computer program instructions stored thereon, when executed by the processor, perform to:
in accordance with a variability tendency of a biological signal having periodicity exhibited in time-series data of the biological signal having periodicity resulting from an abnormality value process performed to eliminate an abnormality value from the biological signal having periodicity in response to an input of the biological signal having periodicity, interpolates a missing value in the biological signal having periodicity in the time-series data of the biological signal having periodicity resulting from the abnormality value process; and
calculates a time feature value focusing on the variability tendency of the biological signal having periodicity measured over an arbitrary time period, from the time-series data of the biological signal having periodicity interpolated by the interpolation unit.

2. The time feature value calculation apparatus according to claim 1, wherein the computer program instructions further perform to
the interpolation unit interpolates the missing value in the biological signal having periodicity, by performing one selected from among: a linear interpolation; an interpolation using a mathematical function approximating the variability tendency of the biological signal having periodicity; and an interpolation using an average value of data sectioned by an arbitrary duration.

3. The time feature value calculation apparatus according to claim 1, wherein the computer program instructions further perform to
determines whether a characteristic of the time feature value to be calculated corresponds to a time feature value focusing on the variability tendency of the biological signal having periodicity measured over the arbitrary time period or to a time feature value focusing on a relationship between adjacent signals each being the biological signal having periodicity,
only when the characteristic of the time feature value to be calculated includes the time feature value focusing on the variability tendency of the biological signal having periodicity measured over the arbitrary time period.

4. The time feature value calculation apparatus according to claim 1, wherein the computer program instructions further perform to
determines whether or not the interpolation by is to be performed, depending on a type of the time feature value which is to be calculated and which focuses on the variability tendency of the biological signal having periodicity measured over the arbitrary time period, and
when the interpolation is not to be performed calculates the time feature value focusing on the variability tendency of the biological signal having periodicity measured over the arbitrary time period, from the time-series data of the biological signal having periodicity resulting from the process to eliminate the abnormality value.

5. The time feature value calculation apparatus according to claim 4, wherein
the biological signal having periodicity is an instantaneous heart rate, examples of the type of the time feature value which is to be calculated by the time feature value calculation unit and which focuses on the variability tendency of the biological signal having periodicity measured over the arbitrary time period include an average heart rate, an average RRI, CVNN, SDNN, and SDANN, and the computer program instructions further perform to
determines that the interpolation is to be performed, when the time feature value to be calculated is one of the average RRI and the average heart rate.

6. A time feature value calculation method for calculating a time feature value of a biological signal having periodicity from time-series data of the biological signal having periodicity, comprising:
interpolating, in accordance with a variability tendency of the biological signal having periodicity exhibited in the time-series data of the biological signal having periodicity resulting from an abnormality value process performed to eliminate an abnormality value from the biological signal having periodicity in response to an input of the biological signal having periodicity, a missing value in the biological signal having periodicity in the time-series data of the biological signal having periodicity resulting from the abnormality value process; and
calculating a time feature value focusing on the variability tendency of the biological signal having periodicity measured over an arbitrary time period, from the interpolated time-series data of the biological signal having periodicity.

7. A program that, when executed by a computer, causes the computer to function as the time feature value calculation apparatus according to claim 1.
